(19) United States
(12) Patent Application Publication
(10) Pub. No.: US 2020/0146586 A1
(43) Pub. Date: May 14, 2020

(54) Creating Accurate Computational Head Models of Patients Using Datasets Combining MRI and CT Images

(71) Applicant: Novocure GmbH, Root D4 (CH)

(72) Inventors: Ariel NAVEH, Haifa (IL); Zeev BOMZON, Kiryat Tivon (IL)

(73) Assignee: Novocure GmbH, Root D4 (CH)

(21) Appl. No.: 16/680,953

(22) Filed: Nov. 12, 2019

Related U.S. Application Data
(60) Provisional application No. 62/760,998, filed on Nov. 14, 2018.

(51) Int. Cl. A61B 5/053; A61B 5/055; A61B 5/00; A61B 6/03; A61B 6/00; A61N 1/40; G06T 17/00

(52) U.S. Cl. CPC A61B 5/0536; A61B 5/055; A61B 5/0042; A61B 6/032; G06T 2210/41; A61B 6/501; A61N 1/40; G06T 17/00; A61B 6/5247

(57) ABSTRACT

A 3D model of AC electrical conductivity of the head (or other body part) can be generated by obtaining both CT and MRI images of the head, and combining the CT and MRI images into a composite model that specifies a conductivity at each voxel of the composite model. Voxels in the composite model that correspond to bone (e.g., the skull) are derived from the CT image, and voxels that correspond to the brain (e.g., white matter, gray matter, etc.) are derived from the MRI image. In some embodiments, the 3D model of conductivity is used to determine the positions for the electrodes in TTFields (Tumor Treating Fields) treatment.

### CROSS REFERENCE TO RELATED APPLICATIONS

This Application claims the benefit of U.S. Provisional Application 62/760,998 (filed Nov. 14, 2018), which is incorporated herein by reference in its entirety.

### BACKGROUND

Tumor Treating Fields, or TTFields, are low intensity (e.g., 1-3 V/cm) alternating electric fields within the intermediate frequency range (100-300 kHz). This non-invasive treatment targets solid tumors and is described in U.S. Pat. No. 7,565,205, which is incorporated herein by reference in its entirety. TTFields are FDA-approved for the treatment of glioblastoma multiforme (GBM), and may be delivered, for example, using the Novocure Optune® system. TTFields are typically delivered using two pairs of transducer arrays positioned on the patient's head to generate perpendicular fields within the treated tumor. More specifically, for the Optune® system, one pair of transducer arrays is located to the left and right (LR) of the tumor, and the other pair is located anterior and posterior (AP) to the tumor.

In-vivo and in-vitro studies show that the efficacy of TTFields therapy increases as the intensity of the electric field increases. Therefore, optimizing array placement on the patient's scalp to increase the intensity in the diseased region of the brain is standard practice for the Optune system. Simulation-based studies have shown that the distribution of TTFields within the brain is heterogeneous and depends on patient anatomy. See, e.g., Miranda P C et al. Phys Med Biol 2014; 59(15):4137-4147; Wenger C et al. Phy Med Biol 2015; 60(18):7339-7357; and Korshoej A R et al. PLoS One 2016; 11(10):e0164051, each of which is incorporated herein by reference in its entirety. So for improved treatment, the transducer arrays' positions may be adapted according to patient-specific head anatomy and tumor location. The transducer arrays' positions, as well as the electrical properties (EPs) of brain tissues, may be used to determine how TTFields distribute within the head.

Array placement optimization may be done using a variety of conventional approaches. One prior art approach is to place the arrays on the scalp as close to the tumor as possible, e.g., using the NovoTal™ system. Another prior art approach is described in U.S. Pat. No. 10,188,851, which is incorporated herein by reference in its entirety. More specifically, the '851 patent discloses determining the electrical conductivity for voxels within the brain using data derived from MRI images, combining that data for the brain with shells that represent the electrical conductivity of the skull and scalp, and subsequently using both the brain data and the skull/scalp shells to form a complete model of the head. This head model is subsequently used to run simulations to determine where to position the transducer arrays so that the intensity of the fields will be sufficiently high in the diseased region of the brain.

Yet another prior art approach is to manually segment an MRI image into various tissue types (e.g., by assigning a tissue type to each voxel), assuming a conductivity for each tissue type, and using the assumed conductivity at each voxel to form a complete model of the head. Here again, the head model is subsequently used to run simulations to determine where to position the transducer arrays.

### SUMMARY OF THE INVENTION

One aspect of the invention is directed to a first method of creating a 3D model of AC electrical conductivity or resistivity of a body part at a given frequency. The first method comprises the steps of obtaining a CT image of the body part; obtaining an MRI image of the body part; and combining the CT image of the body part and the MRI image of the body part into a composite model of the body part that specifies a conductivity or resistivity at each voxel of the composite model. Voxels in the composite model that correspond to bone are derived from the CT image, and voxels in the composite model that correspond to non-rigid tissue are derived from the MRI image.

In some instances of the first method, the combining comprises registering the CT image to the MRI image. In some instances of the first method, segmentation is performed on the CT image prior to the combining. In some instances of the first method, voxels that correspond to metal in the composite model are derived from the CT image. In some instances of the first method, the body part comprises a head, and the non-rigid tissue comprises white matter and grey matter of a brain. In some instances of the first method, the 3D model of AC electrical conductivity or resistivity is a 3D model of AC electrical conductivity.

Another aspect of the invention is directed to a second method of optimizing positions of a plurality of electrodes placed on a subject's body, where the electrodes are used to impose an electric field in a target volume within a body part at a given frequency. The second method comprises the steps of obtaining a CT image of the body part; obtaining an MRI image of the body part, and combining the CT image of the body part and the MRI image of the body part into a 3D composite model of the body part that specifies a conductivity or resistivity at each voxel of the composite model. Voxels in the composite model that correspond to bone are derived from the CT image, and voxels in the composite model that correspond to non-rigid tissue are derived from the MRI image. The second method also comprises the steps of identifying a location of the target volume within the body part; and determining positions for the electrodes based on the composite model and the identified location of the target volume.

Some instances of the second method further comprise the steps of affixing the electrodes to the subject's body at the determined positions; and applying electrical signals between the electrodes subsequent to the affixing, so as to impose the electric field in the target volume.

In some instances of the second method, the combining comprises registering the CT image to the MRI image. In some instances of the second method, segmentation is performed on the CT image prior to the combining. In some instances of the second method, voxels that correspond to metal in the composite model are derived from the CT image. In some instances of the second method, the body part comprises a head, and the non-rigid tissue comprises white matter and grey matter of a brain. In some instances of the second method, the 3D composite model specifies a conductivity at each voxel of the composite model.

### BRIEF DESCRIPTION OF THE DRAWINGS

FIG. 1 is a flowchart of one example for creating a model of a head and optimizing the electric field using that model.

FIG. 2 shows registering a CT scan onto an MRI scan using an affine transformation in order to form a composite image.

FIG. 3 depicts an example of segmenting CT and MRI images before those images are merged into a composite model.

### DESCRIPTION OF THE PREFERRED EMBODIMENTS

The optimization of TTFields treatment may be improved based on an in-depth understanding about how TTFields distribute within the brain. Since measuring field distributions within the patient's brain is extremely difficult, studies addressing this topic often rely on numerical simulations utilizing realistic computational head models. These models are typically derived by segmenting magnetic resonance imaging (MRI) datasets into various tissue types and assigning appropriate dielectric properties to each tissue. But because the visibility of skull defects, bone flaps, and metal plates on MRI scans is low, those features are often ignored or segmented inaccurately within MRI generated models.

This application describes an improved approach for creating realistic head models for simulating TTFields that augments the conventional MRI-based approach using computerized tomography (CT) images. Because skull defects, bone flaps, and metal plates are highly visible in CT images, those features can be identified and outlined from a CT image set, and combined with the images of tissue obtained from the MRI scans (which provides superior anatomical accuracy of non-rigid tissue such as the brain).

This description is divided into two parts: Part 1 describes methods for creating realistic head models for TTFields simulations using both MRI and CT data. Part 2 describes how to optimize TTFields array positions using the model created in part 1.

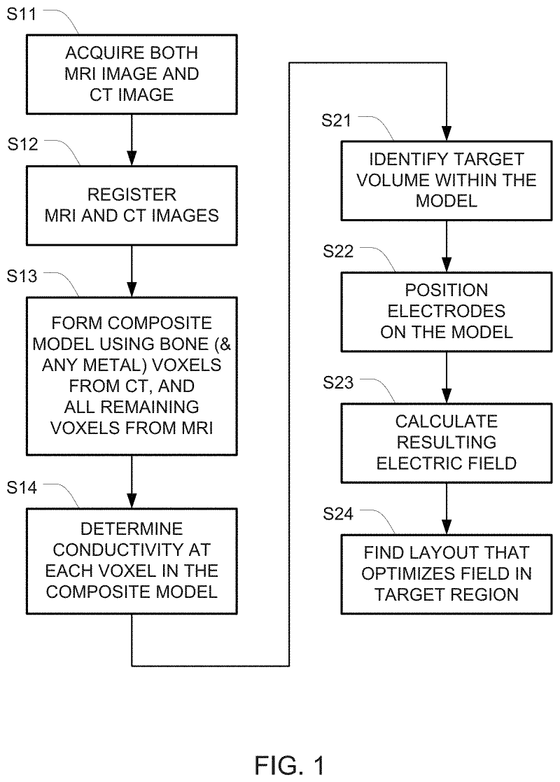

FIG. 1 is a flowchart of one example for creating the model (in steps S11-S14) and optimizing the electric field using that model (steps S21-S24).

Part 1: Creation of a Realistic Computational Phantom Using Both MRI Data and CT Data.

Creating an accurate computational phantom involves accurately mapping the electric properties (e.g., conductivity, resistivity, permittivity) at each point within the computational phantom. Note that while the embodiments described herein discuss mapping conductivity, alternative embodiments can provide similar results by mapping a different electrical property such as resistivity.

Steps S11-S14 in FIG. 1 depict one example of a set of steps that may be used to generate a computational phantom representing a patient based on both MRI and CT images.

Step S11 is the image acquisition step. In this step, both MRI and CT images of the subject that will ultimately be treated using TTFields are obtained. CT provides superior accuracy over MRI in identifying bone, bone flaps, and metal, while MRI provides superior accuracy over CT in identifying non-rigid tissue (including but not limited to gray matter, white matter, and tumor tissue).

In order to create a good computational phantom, high resolution images should be obtained. A resolution of at least 1 mm×1 mm for each slice of both the MRI and CT images, with slice-to-slice spacing of less than 5 mm, is preferable. Lower resolution images may be used for one or both of these types of images, but the lower resolution will yield less accurate phantoms. Optionally, the data set may be inspected and images affected by large artifacts may be removed. Scanner-specific pre-processing may be applied. For example, images may be converted from DICOM format to NIFTI.

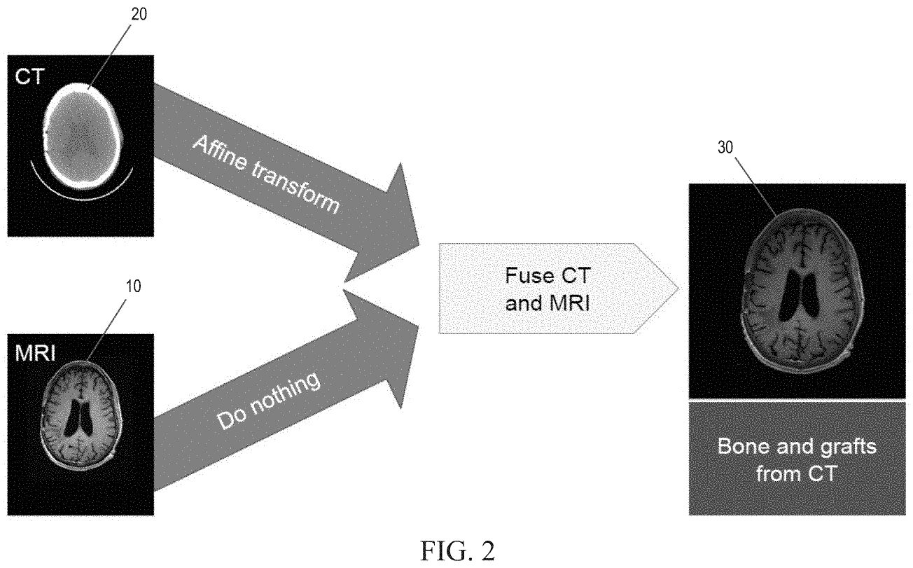

In step S12, the MRI image and the CT image are registered. This may be implemented by registering the MRI image to the CT image; registering the CT image to the MRI image; or registering both the MRI image and the CT image into a standard space (for example the Montreal Neurological Institute, MNI, space) or into any third image. This can be done, for example, using readily available software packages including but not limited to FSL FLIRT, and SPM. MRI scans may also be co-registered with CT scans using 3D Slicer software (www.slicer.org) and any suitable transformation (e.g., simple affine transformations). This is depicted in FIG. 2, which shows registering a CT scan 20 onto an MRI scan 10 using an affine transformation in order to form a composite image 30. This is the first step in creating a combined patient model.

Next, in step S13, a composite model is formed from the post-registration CT and MRI images. Preferably, in the composite model, voxels that correspond to bone (including bone flaps) and metal are derived from the CT image, and voxels that corresponded to gray matter, white matter, tumors and other abnormalities, and CSF are derived from the MRI image.

One approach for forming this composite model is to begin with the MRI image, and overwrite any voxel that, based on data obtained from the CT image, is deemed to be a bone or metal voxel. In some embodiments, the decision of whether each voxel in the CT image is a bone or metal voxel may be made by automatic or manual segmentation of the CT image. In other embodiments, the decision of whether each voxel in the CT image is a bone or metal voxel may be made by comparing the intensity of the voxel to a threshold.

Another approach for forming the composite model is to begin with the CT image and overwrite any voxel that, based on data contained in the CT image, is deemed not to be bone or metal. The decision of whether each voxel in the CT image is a bone or metal voxel may be made using the same approaches described in the previous paragraph. Yet another approach for forming the composite model is to begin with a blank image that is registered with both the CT image and the MRI image. All voxels that correspond to bone or metal from the CT image are marked in the blank image as being either bone or metal, respectively. (Once again, the decision of whether each voxel in the CT image is a bone or metal voxel may be made using the same approaches described in the previous paragraph.) Data for all remaining voxels in the blank image is then copied over from the MRI image.

After the composite model has been formed, processing proceeds to step S14 where the conductivity of each voxel in the composite model is determined. This step may be implemented using any of a variety of alternative approaches. In one approach, the composite model is manually segmented into appropriate categories (e.g., bone, gray matter, white matter, tumor, scar tissue, CSF, metal, etc.) by a human operator.

After a category has been assigned to each voxel in the composite model as described above based on the segmentation, known estimates for the conductivity for each of the categories are used to form a 3D conductivity map). For example, voxels of scar tissue may be assigned an electrical conductivity (a) of 0.36 S/m and a relative permittivity (εᵣ) of 1. And voxels of metal may be assigned the properties of titanium (σ=1.28×10⁶ S/m, εᵣ=1), simulating commonly used medically grafts.

In another approach, the conductivity for each voxel of bone or metal (i.e., those voxels which originated from the CT image) is determined based on manual segmentation of the CT image plus a known estimate for the conductivity of bone and metal. And the conductivity of the remaining voxels is determined directly from the MRI images to form the 3D conductivity map. Examples of suitable approaches for determining conductivity directly from MRI images can be found in U.S. Pat. No. 10,188,851 and US 2019/0308016, each of which is incorporated herein by reference in its entirety.

Note that while the example above describes performing segmentation on the composite model, it is also possible to perform segmentation on the CT and MRI images before those images are merged into the composite model. This may be done, for example, from the T1 post-contrast image using ITK-SNAP (www.itk-snap.com). ITK-SNAP may also be used to manually segment skull defects, metal plates, and metal screws from the CT images. In this case, conductivities may be assigned/determined for each of the skull/metal voxels in the CT image and each of the brain voxels in the MRI image, and the conductivities from those two separate images are subsequently merged into a composite model that specifies a conductivity for each voxel within the composite model (i.e., into a 3D conductivity map).

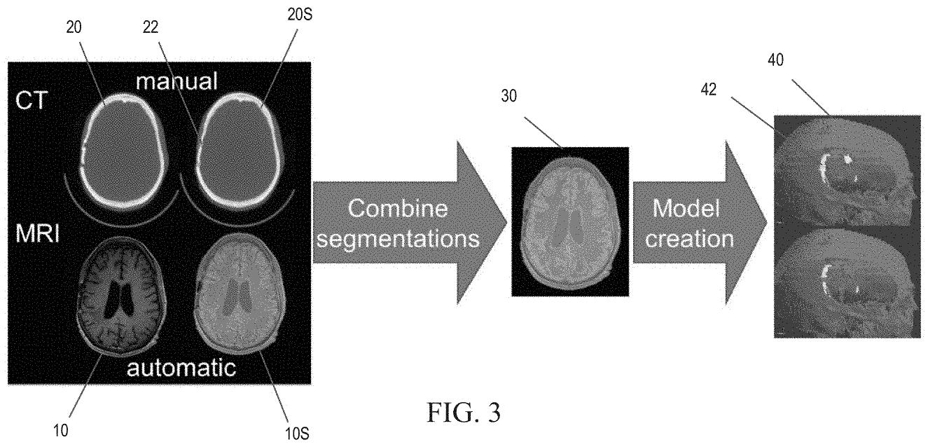

FIG. 3 depicts an example of this approach. Here, an MRI scan 10 is automatically segmented into an output image 10S that includes scalp, grey matter, white matter, and cerebrospinal fluid components; and a CT scan 20 is manually segmented into an output image 20S (e.g., using the Multi-Atlas Robust Segmentation (MARS) tool). Skull defects 22 are visible in the CT image 20S. The tumor location (not shown) is manually segmented from the MRI image 10S. the automatic and manual segmentations from the MRI image 10S and the CT image 20S are combined to yield an intermediate image 30. Finally, conductivities are assigned to each voxel in the intermediate image 30 based on the tissue/material type (i.e., bone, white matter, gray matter, metal, etc.) and a known conductivity for each tissue type to form a composite model 40. Notably, regions 42 in the composite model 40 correspond to the skull defects 22 visible in the CT image 20S.

In any of the embodiments described above, instead of determining the conductivity of voxels that correspond to the scalp based on the MRI images, the scalp may be modeled using a shell that is positioned immediately outside the skull (e.g., with a constant thickness and an assigned conductivity).

Note that in those embodiments where the images were previously registered into a standard space (e.g., in step S12), the model should be subsequently transformed back into patient space (e.g. after step S13 or after step S14).

The 3D conductivity map obtained using any of the approaches described above is subsequently used to optimize the position of the electrodes that are used to apply TTFields to a person's head (by running simulations using the 3D map, as described below in part 2).

Returning to FIG. 1, steps S13 and S14 collectively create a 3D conductivity map from the CT and MRI images. This 3D conductivity map is superior to corresponding maps obtained using prior art approaches because CT scans provide superior detection of skull defects. These defects can be combined with MRI-based segmentation to yield improved 3D head models of glioblastoma patients.

Combining MRI and CT 3D head models will help to elucidate to what extent skull defects, which occur during brain surgery, influence TTFields distribution within the head. This information may be used to implement patient-specific treatment by determining where the optimum positions are for positioning the transducer arrays on each individual patient's head.

Part 2: Optimization of TTFields Array Positions Using Realistic Head Models

Optimization of array layouts means finding the array layout that optimizes the electric field within the target volume (e.g., a tumor or other diseased regions of the patient's brain). This optimization may be implemented by performing the following four steps: (S21) identifying the volume targeted for treatment (target volume) within the realistic head model; (S22) automatically placing transducer arrays and setting boundary conditions on the realistic head model; (S23) calculating the electric field that develops within the realistic head model once arrays have been placed on the realistic head model and boundary conditions applied; and (S24) running an optimization algorithm to find the layout that yields optimal electric field distributions within the target volume. A detailed example for implementing these four steps is provided below.

Step S21 involves locating the target volume within the realistic head model (i.e., defining a region of interest). A first step in finding a layout that yields optimal electric field distributions within the patient's body is to correctly identify the location and target volume, in which the electric field should be optimized.

In some embodiments, the target volume will be either the Gross Tumor Volume (GTV) or the Clinical Target Volume (CTV). The GTV is the gross demonstrable extent and location of the tumor, whereas the CTV includes the demonstrated tumors if present and any other tissue with presumed tumor. In many cases the CTV is found by defining a volume that encompasses the GTV and adding a margin with a predefined width around the GTV.

In order to identify the GTV or the CTV, it is necessary to identify the volume of the tumor within the MRI images. This can be performed either manually by the user, automatically, or using a semi-automatic approach in which user-assisted algorithms are used. When performing this task manually, the MRI data could be presented to a user, and the user could be asked to outline the volume of the CTV on the data. The data presented to the user could be structural MRI data (e.g., T1, T2 data). The different MRI modalities could be registered onto each other, and the user could be presented with the option to view any of the datasets, and outline the CTV. The user could be asked to outline the CTV on a 3D volumetric representation of the MRIs, or the user could be given the option of viewing individual 2D slices of the data, and marking the CTV boundary on each slice. Once the boundaries have been marked on each slice, the CTV within the anatomic volume (and hence within the realistic model) can be found. In this case, the volume marked by the user would correspond to the GTV. In some embodiments, the CTV could then be found by adding margins of a predefined width to the GTV. Similarly, in other embodiments, the user might be asked to mark the CTV using a similar procedure.

An alternative to the manual approach is to use automatic segmentation algorithms to find the CTV. These algorithms perform automatic segmentation algorithms to identify the CTV using the structural MRI data.

Optionally, semi-automatic segmentation approaches of the MRI data may be implemented. In an example of these approaches, a user iteratively provides input into the algorithm (e.g., the location of the tumor on the images, roughly marking the boundaries of the tumor, demarcating a region of interest in which the tumor is located), which is then used by a segmentation algorithm. The user may then be given the option to refine the segmentation to gain a better estimation of the CTV location and volume within the head.

Whether using automatic or semi-automatic approaches, the identified tumor volume would correspond with the GTV, and the CTV could then be found automatically by expanding the GTV volume by a pre-defined amount (e.g., defining the CTV as a volume that encompasses a 20 mm wide margin around the tumor).

Note that in some cases, it might be sufficient for the user to define a region of interest in which they want to optimize the electric field. This region of interest might be for instance a box volume, a spherical volume, or volume of arbitrary shape in the anatomic volume that encompasses the tumor. When this approach is used, complex algorithms for accurately identifying the tumor may not be needed.

Step S22 involves automatically calculating the position and orientation of the arrays on the realistic head model for a given iteration. Each transducer array used for the delivery of TTFields in the Optune™ device comprise a set of ceramic disk electrodes, which are coupled to the patient's head through a layer of medical gel. When placing arrays on real patients, the disks naturally align parallel to the skin, and good electrical contact between the arrays and the skin occurs because the medical gel deforms to match the body's contours. However, virtual models are made of rigidly defined geometries. Therefore, placing the arrays on the model requires an accurate method for finding the orientation and contour of the model surface at the positions where the arrays are to be placed, as well as finding the thickness/geometry of the gel that is necessary to ensure good contact of the model arrays with the realistic patient model. In order to enable fully automated optimization of field distributions these calculations have to be performed automatically.

A variety of algorithms to perform this task may be used, and one such algorithm is described in U.S. Pat. No. 10,188,851, which is incorporated herein by reference in its entirety.

Step S23 involves calculating the electric field distribution within the head model for the given iteration. Once the head phantom is constructed and the transducer arrays (i.e., the electrode arrays) that will be used to apply the fields are placed on the realistic head model, then a volume mesh, suitable for finite element (FE) method analysis, can be created. Next boundary conditions can be applied to the model. Examples of boundary conditions that might be used include Dirichlet boundary (constant voltage) conditions on the transducer arrays, Neumann boundary conditions on the transducer arrays (constant current), or floating potential boundary condition that set the potential at that boundary so that the integral of the normal component of the current density is equal to a specified amplitude. The model can then be solved with a suitable finite element solver (e.g., a low frequency quasistatic electromagnetic solver) or alternatively with finite difference (FD) algorithms. The meshing, imposing of boundary conditions and solving of the model can be performed with existing software packages such as Sim4Life, Comsol Multiphysics, Ansys, or Matlab. Alternatively, custom computer code that realizes the FE (or FD) algorithms could be written. This code could utilize existing open-source software resources such as C-Gal (for creating meshes), or FREEFEM++(software written in C++ for rapid testing and finite element simulations). The final solution of the model will be a dataset that describes the electric field distribution or related quantities such as electric potential within the computational phantom for the given iteration.

Step S24 is the optimization step. An optimization algorithm is used to find the array layout that optimizes the electric field delivery to the diseased regions of the patient's brain (tumor) for both application directions (LR and AP, as mentioned above). The optimization algorithm will utilize the method for automatic array placement and the method for solving the electric field within the head model in a well-defined sequence in order to find the optimal array layout. The optimal layout will be the layout that maximizes or minimizes some target function of the electric field in the diseased regions of the brain, considering both directions at which the electric field is applied. This target function may be for instance the maximum intensity within the diseased region or the average intensity within the diseased region. It also possible to define other target functions.

There are a number of approaches that could be used to find the optimal array layouts for patients, three of which are described below. One optimization approach is an exhaustive search. In this approach the optimizer will include a bank with a finite number of array layouts that should be tested. The optimizer performs simulations of all array layouts in the bank (e.g., by repeating steps S22 and S23 for each layout), and picks the array layouts that yield the optimal field intensities in the tumor (the optimal layout is the layout in the bank that yields the highest (or lowest) value for the optimization target function, e.g., the electric field strength delivered to the tumor).

Another optimization approach is an iterative search. This approach covers the use of algorithm such as minimum-descent optimization methods and simplex search optimization. Using this approach, the algorithm iteratively tests different array layouts on the head and calculates the target function for electric field in the tumor for each layout. This approach therefore also involves repeating steps S22 and S23 for each layout. At each iteration, the algorithm automatically picks the configuration to test based on the results of the previous iteration. The algorithm is designed to converge so that it maximizes (or minimizes) the defined target function for the field in the tumor.

Yet another optimization approach is based on placing a dipole at the center of the tumor in the model. This approach differs from the other two approaches, as it does not rely on solving field intensity for different array layouts. Rather, the optimal position for the arrays is found by placing a dipole aligned with the direction of the expected field at the center of the tumor in the model, and solving the electromagnetic potential. The regions on the scalp where the electric potential (or possibly electric field) is maximal will be the positions where the arrays are placed. The logic of this method is that the dipole will generate an electric field that is maximal at the tumor center. By reciprocity, if we were able to generate the field/voltage on the scalp that the calculation yielded, then we would expect to obtain a field distribution that is maximal at the tumor center (where the dipole was placed). The closest we can practically get to this with our current system is to place the arrays in the regions where the potential induced by the dipole on the scalp is maximal.

Note that alternative optimization schemes can be used to find an array layout that optimizes the electric field within diseased regions of the brain. For example, algorithms that combine the various approaches mentioned above. As an example of how these approaches may be combined, consider an algorithm in combining the third approach discussed above (i.e., positioning the dipole at the center of the tumor in the model) with the second approach (i.e., the iterative search). With this combination, an array layout is initially found using the dipole at the center of the tumor approach. This array layout is used as input to an iterative search that finds the optimal layout.

Once the layout that optimizes the electric field within the diseased regions of the patient's brain has been determined (e.g., using any of the approaches explained herein), the electrodes are positioned in the determined positions. AC voltages are then applied to the electrodes (e.g., as described in U.S. Pat. No. 7,565,205, which is incorporated herein by reference) to treat the disease.

Computational phantoms built in this manner could also be used for other applications in which calculating electric field and or electric current distributions within the head may be useful. These applications include, but are not limited to: direct and alternating current trans-cranial stimulation; simulations of implanted stimulatory electrode field maps; planning placement of implanted stimulatory electrodes; and source localization in EEG.

Finally, although this application describes a method for optimizing array layouts on the head, it could potentially be extended for optimizing array layouts for treatment of other body regions such as the thorax or abdomen.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method of creating a 3D model of AC electrical conductivity or resistivity of a body part at a given frequency, the method comprising the steps of:
obtaining a CT image of the body part;
obtaining an MRI image of the body part; and
combining the CT image of the body part and the MRI image of the body part into a composite model of the body part that specifies a conductivity or resistivity at each voxel of the composite model,
wherein voxels in the composite model that correspond to bone are derived from the CT image, and wherein voxels in the composite model that correspond to non-rigid tissue are derived from the MRI image.

2. The method of claim 1, wherein the combining comprises registering the CT image to the MRI image.

3. The method of claim 1, wherein segmentation is performed on the CT image prior to the combining.

4. The method of claim 1, wherein voxels that correspond to metal in the composite model are derived from the CT image.

5. The method of claim 1, wherein the body part comprises a head, and wherein the non-rigid tissue comprises white matter and grey matter of a brain.

6. The method of claim 1, wherein the 3D model of AC electrical conductivity or resistivity is a 3D model of AC electrical conductivity.

7. A method of optimizing positions of a plurality of electrodes placed on a subject's body, wherein the electrodes are used to impose an electric field in a target volume within a body part at a given frequency, the method comprising the steps of:
obtaining a CT image of the body part;
obtaining an MRI image of the body part; and
combining the CT image of the body part and the MRI image of the body part into a 3D composite model of the body part that specifies a conductivity or resistivity at each voxel of the composite model, wherein voxels in the composite model that correspond to bone are derived from the CT image, and wherein voxels in the composite model that correspond to non-rigid tissue are derived from the MRI image;
identifying a location of the target volume within the body part; and
determining positions for the electrodes based on the composite model and the identified location of the target volume.

8. The method of claim 7, further comprising the steps of:
affixing the electrodes to the subject's body at the determined positions; and
applying electrical signals between the electrodes subsequent to the affixing, so as to impose the electric field in the target volume.

9. The method of claim 7, wherein the combining comprises registering the CT image to the MRI image.

10. The method of claim 7, wherein segmentation is performed on the CT image prior to the combining.

11. The method of claim 7, wherein voxels that correspond to metal in the composite model are derived from the CT image.

12. The method of claim 7, wherein the body part comprises a head, and wherein the non-rigid tissue comprises white matter and grey matter of a brain.

13. The method of claim 7, wherein the 3D composite model specifies a conductivity at each voxel of the composite model.
